(19) United States
(12) Patent Application Publication
(10) Pub. No.: US 2003/0007993 A1
(43) Pub. Date: Jan. 9, 2003

(54) biomedical devices with antimicrobial cationic peptide and protein coatings

(76) Inventors: Mark Wilcox, Balmain NSW (AU); Doug Vanderlaan, Jacksonville, FL (US); Yulina Aliwarga, Bondi Junction (AU); Jenny Lun, Auburn NSW (AU)

Correspondence Address: Audley A. ciamporcero, Jr. Esq., Johnson & Johnson, One Johnson & Johnson Plaza, New Brunswick, NJ, 08933-7003, US

(21) Appl. No.: 09/516,636

(22) Filed: Mar. 1, 2000

(51) Int. Cl. A61F 2/00

(52) U.S. Cl. 424/427

(57) ABSTRACT

Biomedical devices with antimicrobial coatings are provided. One or more surfaces of the device are coated with a cationic peptide, cationic proteins, or mixtures thereof to impart antimicrobial properties to the surface.

### FIELD OF THE INVENTION

This invention relates to coated devices. In particular, the invention provides biomedical devices on the surfaces of which antimicrobial coatings of a cationic peptide, a cationic protein, or both are formed.

### BACKGROUND OF THE INVENTION

Devices for use in and on the human body are well known. The chemical composition of the surfaces of such devices plays a pivotal role in dictating the overall efficacy of the devices. Additionally, it is known that providing such devices with an antimicrobial surface is advantageous.

A wide variety of bactericidal and bacteriostatic coatings have been developed. For example, cationic antibiotics, such as polymyxin, vancomycin, and tetracycline have been used as coatings for contact lenses. Further, metal chelating agents, substituted and unsubstituted polyhydric phenols, aminophenols, alcohols, acid and amine derivatives, and quaternary ammonium have been used as antimicrobial agents for contact lenses.

However, the use of these known antimicrobial coatings has disadvantages. With the use of antibiotic coatings, microorganisms resistant to the antibiotics may develop. Chelating agent use fails to address the numbers of bacteria that adhere to the device. Some of the prior art coatings, for example phenol derivatives and cresols, can produce ocular toxicity or allergic reactions. Quaternary ammonium compounds are problematic because of their irritancy. Thus, a need exists for safe and effective antimicrobial coatings that overcomes at least some of these disadvantages.

### DETAILED DESCRIPTION OF THE INVENTION AND PREFERRED EMBODIMENTS

The present invention provides biomedical devices with an antimicrobial coating and processes for the production of the biomedical devices. It is an unexpected discovery of the invention that certain cationic peptides, cationic proteins, or both may be used to provide antimicrobial coatings for biomedical devices. In particular, it is one discovery of the invention that protamine, melittin, cecropin A, nisin, or combinations thereof, when used as surface coatings, reduce adherence of bacteria to a device's surface, reduce growth of bacteria adhered to a device, or both by greater than about 50 percent.

In one embodiment, the invention provides a biomedical device comprising, consisting essentially of, and consisting of at least one surface comprising, consisting essentially of, and consisting of a coating effective amount of one of protamine, melittin, cecropin A, nisin, or combinations thereof In yet another embodiment, a method for manufacturing biomedical devices comprising, consisting essentially of, and consisting of contacting at least one surface of a biomedical device with a coating effective amount of protamine, melittin, cecropin A, nisin, or combinations thereof is provided.

By “biomedical device” is meant any device designed to be used while in or on either or both human tissue or fluid. Examples of such devices include, without limitation, stents, implants, catheters, and ophthalmic lenses. In a preferred embodiment, the biomedical device is an ophthalmic lens including, without limitation, contact or intraocular lenses. More preferably, the device is a contact lens, most preferably a soft contact lens.

Protamine is isolatable from the sperm of a variety of animals including, without limitation, man. Melittin is isolatable from bee venom. Cecropin A and nisin are isolatable from Aedes aegypti and Lactoccucus lactis, respectively. All four are members of a broad group of cationic peptides and proteins which group includes, without limitation, defensins, magainins, and colicins. It is an unexpected discovery of this invention that only certain cationic peptides and proteins significantly reduce bacterial adherence, bacterial growth, or both on biomedical devices.

Protamine, melittin, cecropin A, and nisin useful in the invention are all commercially available. Alternatively, these cationic peptides and proteins may be produced by known means. For purposes of the invention, generally the purity of the cationic peptide used is at least about 75%, preferably at least about 90%.

Protamine, melittin, cecropin A, nisin, or combinations thereof may be adsorbed to polymer surfaces of a biomedical device. The cationic peptides and proteins may be used on any surface, but most advantageously are used with negatively charged surfaces.

The cationic peptides and proteins alternatively may be bound to the polymer surfaces. This may be either a direct reaction or, preferably, a reaction in which a coupling agent is used. For example, a direct reaction may be accomplished by the use of a reagent of reaction that activates a group in the surface polymer or the cationic peptide making it reactive with a functional group on the peptide or polymer, respectively, without the incorporation of a coupling agent. For example, one or more amine groups on the peptide may be reacted directly with isothiocyanate, acyl azide, N-hydroxysuccinimide ester, sulfonyl chloride, an aldehyde, glyoxal epoxide, carbonate, aryl halide, imido ester, or an anhydride group on the polymer. In an alternative embodiment, coupling agents may be used. Coupling agents useful for coupling the cationic peptide or protein to the device's surface include, without limitation, N,N′-carbonyldiimidazole, carbodimides such as 1-ethyl-3-(3-dimethylaminopropyl)carbodiimide (“EDC”), dicyclohexyl carbodiimide, 1-cylcohexyl-3-(2-morpholinoethyl)carbodiimide, diisopropyl carbodiimide, or mixtures thereof. The carbodiimides also may be used with N-hydroxysuccinimide or N-hydroxysulfosuccinimide to form esters that can react with amines to form amides.

Amino groups also may be coupled to the polymer by the formation of Schiff bases that can be reduced with agents such as sodium cyanoborohydride and the like to form hydrolytically stable amine links. Coupling agents useful for this purpose include, without limitation, N-hydroxysuccinimide esters, such as dithiobis(succinimidylpropionate), 3,3′-dithiobis(sulfosuccinimidylpropionate), disuccinimidyl suberate, bis(sulfosuccinimidyl) suberate, disuccinimidyl tartarate and the like, imidoesters, including, without limitation, dimethyl adipimate, difluorobenzene derivatives, including without limitation 1,5-difluoro-2,4-dinitrobenzene, bromofunctional aldehydes, including without limitation gluteraldehyde, and bis epoxides, including without limitation 1,4-butanediol diglycidyl ether. One ordinarily skilled in the art will recognize that any number of other coupling agents may be used depending on the functional groups present on the device's surface.

One ordinarily skilled in the art also will recognize that, if the device's surface does not contain suitable reactive groups, such suitable groups may be incorporated into the polymer by any conventional organic synthesis methods. Alternatively, the reactive groups may be introduced by the addition of polymerizable monomers containing reactive groups into the monomer mixture used to form the polymer.

Examples of polymer surfaces onto which the cationic peptides and proteins may be adsorbed or bonded are surfaces formed from, without limitation, polymers and copolymers of styrene and substituted styrenes, ethylene, propylene, acrylates and methacrylates, N-vinyl lactams, acrylamides and methacrylamides, acrylonitrile, acrylic and methacrylic acids as well as polyurethanes, polyesters, polydimethylsiloxanes and mixtures thereof Such polymers may include hydrogels and silicone containing hydrogels. Preferably, lightly crosslinked polymers and copolymers of 2-hydroxyethylmethacrylate (“HEMA”) are used. By “lightly crosslinked” is meant that the polymer has a low enough crosslink density so that it is soft and elastic at room temperature. Typically, a lightly crosslinked polymer will have about 0.1 to about 1 crosslinking molecule per about 100 repeating monomer units. Examples of suitable lightly crosslinked HEMA polymers and copolymers include without limitation, etafilcon A and copolymers of glycerol methacrylate and HEMA. Also preferably, silicone hydrogels, especially those of hydrophilic monomers, such as N,N-dimethylacrylamide, are used.

In one embodiment of the process for making the device of the invention, the surface to be coated is contacted with the protamine, melittin, cecropin A, nisin or combinations thereof in any convenient manner. Preferably, mixtures of protamine and melittin are used. For example, the device may be placed in a solution of protamine and solvent into which the medical device is placed. As an alternative, the device's surface may first be treated with a coupling agent and the surface then placed in a solution of the selected cationic peptide or protein. As yet another alternative the peptide or protein may be reacted alone with the polymer surface.

Suitable solvents for use in the invention are those that are capable of dissolving protamine, melittin, cecropin A, or nisin singly or in combination. Preferably, the coating process is carried out in water, alcohol, or mixtures thereof EDC is effective in aqueous solutions and, thus, is a preferred coupling agent.

The coupling agents may be used alone or in combination with agents capable of stabilizing any reactive intermediate formed. For example, EDC may be used with N-hydroxysuccinimide as a stabilizer. Additionally, it may be desirable to adjust the pH. Preferably, the pH is adjusted to about 6.5 to about 8.0, more preferably about 7.0 to about 7.5.

A coupling effective amount of a coupling agent is used which amount is an amount sufficient to couple the peptide or protein to the device surface. The precise amount of coupling agent used will depend on the surface's chemistry as well as the agent selected. Generally, about 0.01 to about 10 weight percent, preferably about 0.01 to about 5.0, more preferably about 0.01 to about 1 weight percent of the coupling agent is used based on the weight of the coating solution. By coating solution is meant the peptide or protein with one or more of the solvent, coupling agent, buffer, and the like. Typically, the amount of coating solution used per lens will be about 1 ml to about 10 ml, preferably about 1 ml to about 5 ml, more preferably about 1 ml to about 2 ml per lens.

In the processes of the invention, a coating effective amount of protamine, melittin, cecropin A, nisin, or combinations thereof is used meaning an amount that when contacted with the surface is sufficient to coat the surface so as to impart the desired antimicrobial properties to the surface. By antimicrobial properties is meant either or both the ability to significantly reduce, meaning by greater than about 50 percent, either or both the amount of bacteria adhering to the surface and the growth of bacteria adhered to the surface. In the case of contact lenses, generally, the amount contacted with the lens is about 1 μg to about 10 mg, preferably about 10 μg to about 1 mg per lens. The amount of coating resulting per contact lens is about 50 to about 1000 μg. In cases in which combinations of melittin and protamine are used, the amount of protamine used preferably is about 500 μg/ml or less.

Temperature and pressure are not critical to the processes of the invention and the process may be conveniently carried out at room temperature and pressure. The contact time used will be a length of time sufficient to coat the surface to the extent desired. Preferably, contact time is about 60 seconds to about 24 hours.

Following contacting, the surface may be washed with water or buffered saline solution to remove unreacted protamine, melittin, colicin and solvent. One ordinarily skilled in the art will recognize that the polymer for producing the surface to be coated by the method of the invention may contain other monomers and additives. For example, ultra-violet absorbing monomers, reactive tints, processing aids, and the like may be used.

The invention will be further clarified by a consideration of the following, non-limiting examples.

### EXAMPLES

In the following examples, the cationic proteins and peptides listed on Table 1 were used.

TABLE 1

| CATIONIC PEPTIDE/PROTEIN | SOURCE |
|---|---|
| Protamine | Fish |
| Cecropin A | Insect |
| Cecropin P1 | Pig |
| Melittin | Insect |
| Melittin-1-13 AA | Synthetic |
| Magainin 1 | Frog |
| Magainin 2 | Frog |
| Defensin HNP-1 | Human |
| β Defensin 1 | Human |
| Secretory leukocyte protease inhibitor (SLPI) | Human |
| Colicin | Gram − bacteria |
| Nisin | Gram + bacteria |

Bacterial strains used in the Examples are listed on Table 2.

TABLE 2

| Strain | Isolation Site |
|---|---|
| Pseudomonas aeruginosa Paer1 | CLARE* |
| Pseudomonas aeruginosa 6294 | MK*-ULCER |
| Pseudomonas aeruginosa 6206 | MK-ulcer |
| Pseudomonas aeruginosa ATCC 15442 | Environmental strain |
| Serratia marcesens Smar5 | CLARE** |
| Escherichia coil Ecol8 | CLARE |
| Staphylococcus intermedius Sint 2 | Asymptomatic lens |
| Staphylococcus aureus Saur31 | CLPU*** |

*By “MK” is meant microbial keratitis.
**By “CLARE” is meant contact lens induced red eye.
***By “CLPU” is meant contact lens-induced peripheral ulcers.

The majority of testing was performed with strains P. aeruginosa 6294 and S. aureus 31. P. aeruginosa and S. aureus are the most common bacteria causing eye inflammation or infections for contact lens wearers. Other strains were used to validate results or assess the effectiveness of the compounds over a range of bacteria.

### Example 1

To assess the effect of the cationic proteins/peptides in solution against rapid growing bacterial cells, bacteria were cultured in Trypticase soy broth (“TSB”) overnight at 35° C. Aliquots (20 μl) of this cell suspension were then added to fresh TSB (10 ml). Different concentrations of the cationic proteins/peptides were added to the fresh broth and incubated for up to 48 h at 35° C. Samples were taken at different time points and the optical density at 660 nm measured as a measure of changes in bacterial numbers was measured.

To assess the effect of the cationic proteins/peptides in solution against slow growing bacterial cells, cells were grown as previously in TSB. The cells were then harvested by centrifugation and washed in phosphate buffered saline (PBS; NaCl 8 g/l; KCl 0.2 g/l; Na₂HPO₄ 1.15 g/l; KH₂PO₄ 0.2 g/l). The cells were then re-suspended to OD 0.1 (unless otherwise stated) at 660 nm in PBS, different concentrations of cationic proteins/peptides were added and incubated for up to 48 h at 35° C. Samples were taken a different points and numbers of bacteria analyzed using the Miles and Misra technique (i.e. numbers that are viable after plating dilutions onto nutrient agar plates).

The results obtained in solution for all cationic proteins and peptides studied are shown on Table 3. Most protein/peptides were screened against only P. aeruginosa 6294 and S. aureus 31. If these showed reductions in growth then other strains may have been examined. As can been seen from Table 3, protamine and melittin were the most efficacious at preventing the growth of gram-positive and gram-negative bacteria in solution.

In Table 3, slow growing cells are those re-suspended in PBS with cationic protein/peptide and rapid growing are those re-suspended in TSB plus cationic protein/peptide. The numbers in μg/ml are in concentration showing peak activity. A “−” sign indicates no reduction in bacterial growth, a “+” sign indicates a 1 to 50% reduction in growth, a “++” sign indicates a 51 to 89% reduction in growth, a “+++” sign indicates a 90 to 98% reduction in growth a “++++” sign indicates a greater than 98% reduction in growth.

TABLE 3

| | Paer 1 | 6294 | 6206 | Saur 31 | Sint 2 | Smar 5 | Ecol 8 |
|---|---|---|---|---|---|---|---|
| Protamine | ++++(slow | ++++(slow | ++++(slow | ++++(slow | ++++ (slow | ++++(1000 | ND* |
| 125- | growing) | growing) | growing) | growing) | growing) | μg/ml slow | |
| 1000 μg/ml | −(rapid | | | | −(rapid | growing) | |
| | growing) | | | | growing) | −(<1000 | |
| | | | | | | μg/ml slow | |
| | | | | | | growing) | |
| Melittin | ++++(24 h, | ++(24 h, | ND | ++++ | ++++(24 h, | — | ND |
| 15 μg/ml | 15 μg/ml | 15 μg/ml | | (15 μg/ml | 15 μg/ml | | |
| | slow | slow | | rapid | slow | | |
| | growing) | growing) | | growing) | growing) | | |
| | −(48 h rapid | −(48 h slow | | ++++(24 h, | ++++(24 h, | | |
| | growing) | growing) | | 15 μg/ml | 15 μg/ml | | |
| | | | | slow | rapid | | |
| | | | | growing) | growing) | | |
| Magainin | — | ++(slow | ND | — | — | ND | ND |
| 1 10- | | growing) | | | | | |
| 200 μg/ml | | | | | | | |
| Magainin | ND | +++(24 h, | ND | — | ND | ND | ND |
| 2 100- | | 400 μg/ml, | | | | | |
| 400 μg/ml | | slow | | | | | |
| | | growing) | | | | | |
| | | −(48 h, | | | | | |
| | | 400 μg/ml, | | | | | |
| | | rapid | | | | | |
| | | growing) | | | | | |
| Cecropin | +(slow | — | ND | −/+(rapid | — | ND | ND |
| P1 1- | growing) | | | growing) | | | |
| 50 μg/ml | −(rapid | | | −(slow | | | |
| | growing) | | | growing) | | | |
| Cecropin | ND | ++++(slow | ND | — | ND | ND | ND |
| A 4- | | growing) | | | | | |
| 60 μg/ml | | | | | | | |
| SLPI 10- | — | — | — | — | — | ND | ND |
| 100 μg/ml | | | | | | | |
| Colicin 1 | ND | +(24 h) | ND | ++(24 h) | ND | ND | −(24 h) |
| 10 units/ml | | | | | | | |
| Nisin | ND | ++(2 μg/ml) | ND | — | ND | ND | ND |
| 32 μg/ml | | | | | | | |
| β Defensin | ND | ++(24h, | ND | — | ND | ND | ND |
| 60 μg/ml | | 60 μg/ml) | | | | | |
| Melittin 1 | ND | ++(24h, | ND | ++(48h, | ND | ND | ND |
| 13- | | 1000 μg/ml) | | 24h, | | | |
| 1000 μg/ml | | | | 1000 μg/ml) | | | |
| Defensin | ND | +(2 μg/ml) | ND | — | ND | ND | ND |
| HNP1 1- | | | | | | | |
| 10 μg/ml | | | | | | | |
| Transferrin | ND | ++ | ND | — | ND | ND | ND |
| 125- | | | | | | | |
| 2000 μg/ml | | | | | | | |

*By “ND” is meant not determined.

### Example 2

For conducting total counts, etafilcon A lenses were removed from the manufacturers vials, washed three times in 1 ml PBS and then coated with various concentrations of cationic proteins/peptides overnight at 37° C. either individually or in combination. After incubation, the lenses were washed three times in PBS and 0.5 ml of 1×10⁸ bacterial cells/ml was added to the lenses. After incubation at ambient temperature for 10 min, the lenses were washed three times in PBS to remove non-adherent or loosely adherent bacteria and stained with crystal violet for 1 min. The number of cells per lens was examined under the microscope. Five grids (0.005625 mm²) per lens were counted and triplicate lenses for each treatment were assayed.

For conducting viable counts, etafilcon A lenses were removed from the manufacturers vials, washed three times in 1 ml PBS and then coated with various concentrations of cationic proteins/peptides overnight at 37° C. (either individually or in combination). After incubation, the lenses were washed three times in PBS and 0.5 ml of 1×10⁸ bacterial cells/ml was added to the lenses. After incubation at ambient temperature for 10 min, the lenses were washed three times in PBS to remove non-adherent or loosely adherent bacteria. Lenses were then homogenized using 1 ml PBS and a small magnetic stirring bar (octagonal cross-section, 0.5″×0.125″) and stirred at maximum speed for one hour which was sufficient for lens disintegration. Serial dilutions were then made according to the technique of Miles and Misra and aliquots (20 μL) plated out on nutrient agar. After incubation overnight at 37° C., viable bacteria were determined and results expressed as colony forming units/mm² after calculation of the surface area of the lens (approximately 310 mm²).

The lenses were incubated in concentrations of cationic proteins/peptides that were either effective in solution or the highest concentration available if there was no effect in solution. After rinsing, bacteria were added and numbers of cells analyzed as the total cells per mm² of the lens or the number of viable cells per mm² of the lens. The results are shown on Table 4.

TABLE 4

| | | Reduction v. | Reduction v. |
|---|---|---|---|
| Peptide | Strain | control* | total** |
| Protamine | 6294 | 80% | 80% |
| | Saur 31 | — | — |
| Melittin | 6294 | 70% | |
| | Saur 31 | 60% | |
| Magainin 1 | 6294 | — | — |
| | Saur 31 | — | — |
| Magainin 2 | 6294 | — | — |
| | Saur 31 | — | — |
| Cecropin P1 | 6294 | — | — |
| | Saur 31 | — | — |
| Cecropin A | 6294 | — | 93% |
| | Saur 31 | — | — |
| SLPI | 6294 | — | — |
| | Saur 31 | — | — |
| Colicin | 6294 | — | — |
| | Saur 31 | — | |
| Nisin | Saur 31 | — | 50% |
| β Defensin 1 | 6294 | — | — |
| | Saur 31 | — | — |
| Melittin-1-13 AA | 6294 | — | — |
| | Saur 31 | — | — |
| Defensin HNP-1 | 6294 | — | — |
| | Saur 31 | — | — |
| Protamine 1000 μg/ml | 6294 | 90% | — |
| and Melittin 15 μg/ml | Saur 31 | — | — |
| Protamine 500 μg/ml | 6294 | 65% | — |
| and Melittin 15 μg/ml | Saur 31 | 60% | — |
| Transferrin (125 μg/ml) | 6294 | — | — |

*Effect compared to adhesion control lens that was not coated with cationic protein/peptide before bacterial adhesion.
**Effect compared to the number of total bacterial cells adhered to lens coated with cationic proteins/peptides, i.e. the cationic prevented the growth of the adhered bacteria even though there may have been an increase in total cell numbers.
“—” indicates no reduction in adhesion.

The results on Table 4 show that although nisin and cecropin A did not reduce the total adhesion of bacteria by increasing the total number of cells on the lens, there was a significant reduction in the viability of those cells compared to the cells adhered to the uncoated lens. This indicates that the adhered bacteria were prevented from growing. Protamine significantly reduced the adhesion of P. aeruginosa 6294 to the lenses and also reduced the viability of the cells on the coated lenses. Melittin both reduced initial adhesion of S. aureus 31. A similar effect was seen for P. aeruginosa 6294 and when a mixture of protamine and melittin was used.

What is claimed is:

1. A device comprising a biomedical device at least one surface of which comprises a coating effective amount of one of protamine, melittin, cecropin A, nisin, or a combination thereof.

2. The device of claim 1 wherein the biomedical device is a contact lens.

3. The device of claim 1, wherein the at least one surface comprises a coating effective amount of protamine.

4. The device of claim 1, wherein the at least one surface comprises a coating effective amount of melittin.

5. The device of claim 1, wherein the at least one surface comprises a coating effective amount of protamine and melittin.

6. The device of claim 1, whereon the at least one surface comprises a coating effective amount of cecropin A.

7. The device of claim 1, whereon the at least one surface comprises a coating effective amount of nisin.

8. The device of claim 1, wherein the surface further comprises a polymer selected from the group consisting of hydrogels, silicone containing hydrogels, polymers and copolymers of 2-hydroxyethylmethacrylate and mixtures thereof

9. The device of claim 8 wherein the polymer is a hydrogel.

10. The device of claim 8 wherein the polymer is a silicone containing hydrogel.

11. The device of claim 8 wherein the polymer is a polymer or copolymer of 2-hydroxyethylmethacrylate.

12. The device of claim 11 wherein the copolymer of 2-hydroxyethylmethacrylate is a lightly crosslinked copolymer of 2-hydroxyethylmethacrylate.

13. A contact lens at least one surface of which comprises a coating effective amount of protamine, melittin, cecropin A, nisin, or a combination thereof.

14. The contact lens of claim 13 wherein the surface further comprises a polymer selected from the group consisting of hydrogels, silicone containing hydrogels, polymers and copolymers of 2-hydroxyethylmethacrylate and mixtures thereof

15. The contact lens of claim 14 wherein the polymer is a hydrogel.

16. The contact lens of claim 14 wherein the polymer is a silicone containing hydrogel.

17. The contact lens of claim 14 wherein the polymer is a polymer or copolymer of 2-hydroxyethylmethacrylate.

18. The contact lens of claim 17 wherein the copolymer of 2-hydroxyethylmethacrylate is a lightly crosslinked copolymer of 2-hydroxyethylmethacrylate.

19. A process for manufacturing a device comprising the step of contacting at least one surface of a biomedical device with a coating effective amount of protamine, melittin, cecropin A, nisin, or a combination thereof.

20. The process of claim 19 wherein the biomedical device is a contact lens.

21. The process of claim 20, further comprising the step of contacting the at least one surface with a coupling effective amount of a coupling agent.

22. The process of claim 20, wherein the at least one surface is contacted with a coating effective amount of protamine.

23. The process of claim 20, wherein the at least one surface is contacted with a coating effective amount of melittin.

24. The process of claim 20, wherein the at least one surface is contacted with a coating effective amount of protamine and melittin.

25. The process of claim 20, wherein the at least one surface is contacted with a coating effective amount of cecropin A.

26. The process of claim 20, wherein the at least one surface is contacted with a coating effective amount of nisin.
